(19) United States
(12) Patent Application Publication
(10) Pub. No.: US 2005/0283164 A1
(43) Pub. Date: Dec. 22, 2005

(54) Apparatus and method for implanting intraocular lens through a small incision

(76) Inventors: Henry Wu, Diamond Bar, CA (US); Christopher D. Wilcox, Mission Viejo, CA (US); Loi Diep, Orange, CA (US)

Correspondence Address: FROST BROWN TODD, LLC, 2200 PNC CENTER, 201 E. FIFTH STREET, CINCINNATI, OH, 45202, US

(21) Appl. No.: 11/145,150

(22) Filed: Jun. 3, 2005

Related U.S. Application Data
(60) Provisional application No. 60/580,916, filed on Jun. 18, 2004.

(51) Int. Cl. A61F 9/00

(52) U.S. Cl. 606/107

(57) ABSTRACT

Apparatus and method for implantation of intraocular lenses (IOLs) with various designs, particularly a full-size IOL, into the eye through small incisions are disclosed. A full-size intraocular lens mimics the natural human crystalline lens in size and volume. The SMART™ IOL, a full-size design, utilizes the thermodynamic properties of a crystalline polymeric material composition. The insertion apparatus and method of the present invention allow the full-size SMART™ IOL to be inserted through a small incision (about 4 mm or less) by deforming it with a crimping device into a solid rod and delivering the rod into the eye with a temperature-controlled injector equipped with a temperature control device. Once the solid rod is located in the desired position inside the eye, human body temperature softens the rod and allows it to reform back into the original lens geometry with defined optical properties.

### CROSS-REFERENCE TO RELATED APPLICATION

This application claims priority from and is based on U.S. Provisional Application No. 60/480,916, Wu et al., filed Jun. 28, 2004, incorporated herein by reference.

### BACKGROUND OF THE INVENTION

The present invention relates to an apparatus for deforming, packaging, and inserting an IOL, especially a full-size IOL, into an eye through a small incision. It also relates to a method for inserting a thermodynamic shape memory intraocular lens into an eye. In specific aspects, the invention relates to a crimper for deforming an IOL into a rod shape, a package device for maintaining the deformed lens in the rod shape until use, and an apparatus with temperature control for inserting a thermodynamic intraocular lens into an eye. This invention also relates to methods for using these apparatus for the purpose of implanting a thermodynamic IOL, whether a full-size design or other designs, into an eye through a small incision.

The current state-of-the-art IOLs, either a three-piece design or single piece design, can be implanted through an incision size of 3 mm or slightly less because they have an optical body with a diameter of about 4.5 to about 6.5 mm and central lens thickness of about 0.4 to about 1.5 mm. When folded, a three-piece IOL has an intersectional dimension of about 3 mm, thus it is possible to implant through a 3 mm incision. A substantial number of instruments have been proposed to aid in inserting such a foldable lens into the eye. They include specialized folding forceps and injection systems. Folding forceps have specially designed tips that essentially fold lenses in half to reduce the diameter of the IOL, allowing them to be implanted through an incision smaller than the original lens diameter. Injectors have a specially designed cartridge (see FIG. 8) to hold the folded lens therein and a plunger to push the folded lens through the cartridge.

On the other hand, a full-size lens, mimicking the natural human crystalline lens with a diameter in the range of 8 to 11 mm and a central lens thickness in the range of 2 to 5 mm, cannot be inserted through a 3 mm incision by simply folding the lens. This is because a full-size lens has a volume 3 to 10 times as large as that of a three-piece IOL. Use of existing lens folding devices would require an incision size of at least 5 mm. Such a large incision size results in other complications, such as longer healing time, induced astigmatism, and an increased potential for intraocular infection. It is preferred that the full size IOL be implanted through a small incision of no longer than about 4 mm, more preferably about 3 mm or less.

The requirement for a small size incision is especially important for a truly accommodative lens. The SMART™ IOL (Medennium, Irvine, Calif.) has the potential for restoring accommodation because of its full-size nature and because of its soft gel-like properties. In the classic Helmholtz theory of accommodation, the capsular bag participates in changing the shape of the lens through the action of the ciliary body and zonules. A small diameter implant will allow implantation using a small hole in the capsular bag, known as a capsulorhexsis. A small hole gives a better chance that the capsule will retain some ability to hold and reshape the implant therein. A large incision lens will require a large capsulorhexsis and consequently it may compromise the eye's capability to change the IOL shape through the interaction between the IOL and capsule.

The desire to implant foldable intraocular lenses through ever smaller incisions has prompted the use of injection systems that will deform lenses into smaller shapes than folding forceps and deliver the deformed lens through a cylindrical tip into the eye. Currently available injection systems have been designed to deform and deliver the current state-of-the-art IOLs. They are not capable of being used for a full size lens, such as the SMART™ IOL. These lens injection systems are also not designed to work with thermodynamic materials, which require a mechanism for controlling the temperature of the lens implant.

Accordingly, it is highly desirable to provide apparatus and methods for deforming, packaging, and implanting IOLs made from thermodynamic materials through a small incision, particularly a full-size lens, which has the potential to be an accommodative IOL.

### SUMMARY OF THE INVENTION

A new apparatus and method for deformation of IOLs (particularly those made from thermodynamic polymeric composition) into a rod shape, packaging the deformed lens in the rod shape, and delivery of the rod into the eye through a small incision constitute parts of the present invention. The apparatus used for deforming an IOL, such as a full-size lens, into a rod shape is a crimper of the type disclosed in U.S. Pat. No. 6,629,350, Motsenbocker, issued Oct. 7, 2003, incorporated by reference herein, as shown in FIG. 1, made from either plastic materials, stainless steel, titanium, ceramic, or other rigid materials. Once the full-size IOL is deformed into a rod shape, it can be placed in a package comprising a tubular channel. The rod is kept inside the tubular channel to prevent the rod from prematurely reverting back to its original lens shape. Therefore, it can be shipped to a doctor's office without shape recovery. Prior to use, the package can be chilled in a refrigerator to ensure the rod will not revert back to its lens shape once the rod is removed from the package. The rod is loaded into an injector, which has a temperature control device to ensure the rod remains in the rod shape until it reaches the desired position inside the eye.

Specifically, the present invention relates to a method for insertion of a thermodynamic shape memory intraocular lens into an eye, comprising the steps of:
(a) placing said lens into a segmental radial compression device;
(b) forming said lens into a rod having a predetermined cross-sectional diameter of from about 1 mm to about 4 mm, using said compression device; and
(c) inserting said lens, configured as a rod, into the eye through an incision.

The present invention also relates to a device for forming a thermodynamic shape memory intraocular lens into a rod for insertion into an eye, comprising a segmental radial compression device holding said thermodynamic shape memory intraocular lens.

Finally, the present invention relates to a device for inserting an intraocular lens configured in the form of a rod through an incision into an eye, said device comprising a central channel to hold the lens in rod form; a sleeve surrounding the central channel, said sleeve holding coolant used to keep the lens at a temperature below its melting temperature; and a plunger used to push the lens (in rod form) out of the central channel and into the eye.

### BRIEF DESCRIPTION OF THE DRAWINGS

FIG. 1 is a view of the SMART™ IOL placed on the surface of a thermoplastic sheet.

FIG. 2 is a view of the SMART™ IOL wrapped within the thermoplastic sheet, prior to deformation.

FIG. 3 is a front view of the crimping system described in U.S. Pat. No. 6,620,350.

FIG. 4 is a side view of the deformed SMART™ IOL rod.

FIG. 5 is a front view of the open channel in a carrier.

FIG. 6 is a front view of a carrier showing the tear away top section.

FIG. 7 is a top view of a channel left in the carrier after the tear away top has been removed.

FIG. 8 is a side view of a thermoplastic cartridge used for SMART™ IOL rod delivery.

FIG. 9 is a side view of an injector device with a flow-through cooling jacket.

### DETAILED DESCRIPTION OF THE INVENTION

In order to better understand the teachings of the present invention, the preferred embodiments given below use the full-size SMART™ IOL for illustration purpose. It is not intended to limit the scope of the present invention. As matter of fact, the current state-of-the-art IOL is not a full-size design; rather, it is a three-piece IOL. Example 3 of the present application illustrates that a three-piece IOL made from thermodynamic polymeric compositions has also been successfully deformed and delivered through an incision of about 2 mm.

The lenses used in the present invention are made from thermodynamic shape memory materials, such as those described in U.S. Pat. No. 6,679,605, Zhou et al., issued Jan. 20, 2004, incorporated herein by reference.

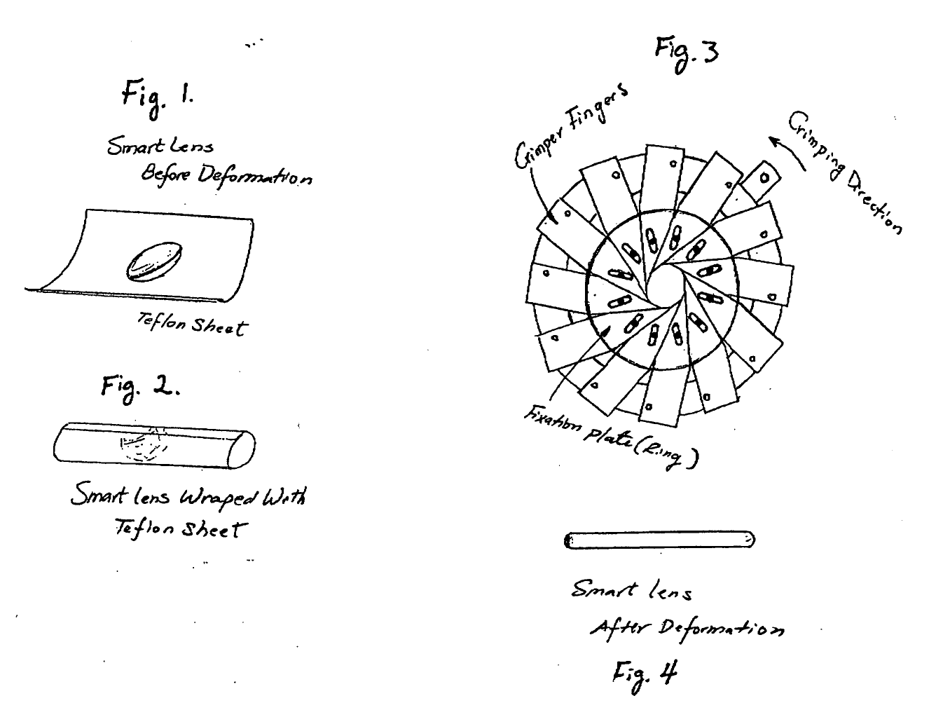

The method for SMART™ IOL deformation includes (1) using a crimper to deform the Smart IOL, (2) using a thermoplastic sheet to protect the SMART™ IOL surface from being damaged by the crimper, and (3) using viscoelastic agents as a lubricant to further protect and separate the SMART™ IOL surface from the thermoplastic sheet. The SMART™ IOL is made from a polymeric composition (preferably crosslinked) with a glass transition temperature at 20° C. or lower (preferably 0° C. or lower) and a melting temperature in the range of from about 1° C. to about 37° C. Above its melting temperature, the SMART™ IOL is a soft, rubbery material while below its melting temperature it is a hard, rigid solid. The method for SMART™ IOL deformation further utilizes the temperature change to deform the lens into a rod shape, to “freeze” it in the rod shape, and to reform the rod back to the lens shape. A thermoplastic sheet, preferably one with low surface energy, such as polytetrafluoroethylene, polyethylene, polypropylene, or polysiloxane, can wrap the SMART™ IOL inside a 10 mm diameter roll. The low surface energy surface will not adhere to the lens material and also provides a low friction surface during the compression process. The sheet acts to protect the optic surface from damage without adhering to the optic surface. See FIG. 1 and FIG. 2. Viscoelastic lubricants, such as aqueous solutions of sodium hyaluronate, hydroxypropylmethyl cellulose or chondroitin sulfate, may be applied to the optic surfaces as additional protection for the optic before enclosing the lens within the plastic film. After the SMART™ IOL is warmed up to a temperature higher than its melting temperature, it is placed in a deformation device, such as a crimper, for example, the segmental radial compression crimper described U.S. Pat. No. 6,629,350, Motsenbocker, issued Oct. 7, 2003, incorporated herein by reference (see FIG. 3). The deformation device compresses the 10 mm diameter optic into a roll having a pre-determined diameter of from about 1 to about 4 mm by a gradual and even radial application of force.

This type of segmental radial compression device has been used to reduce the diameter of cylindrical objects such as stents to allow implantation through the small incisions required for minimally invasive surgery. The crimper has also been used to swage marker bands, another cylindrical device, onto shafts.

However, this type of crimping mechanism has not previously been used for compressing soft intraocular lenses into rods. See FIG. 4. The segmental radial mechanism is capable of accommodating a wide variety of object diameters and the length of the crimper finger face can be designed to accommodate a variety of final rod lengths. This flexibility is important when working with a full-size IOL or a state-of-the-art three piece or single piece IOL. Such lenses are designed with a range of different powers (diopters) to restore or improve vision in people ranging from high myopes to high hyperopes. This range of lenses may have varying diameters, central thicknesses, and, therefore, volumes. As a result, the rod made from lenses with various volumes will vary in diameter and length. Physicians will prefer the smallest possible rod diameter to allow introduction through the smallest possible incision. The smaller the rod diameter, the longer the rod becomes. The rod length for a high power lens or a lens with a diameter greater than about 10 mm may exceed about 35 mm in length. The lens material will have a strain limit that sets a lower limit for a rod diameter to produce undamaged rods that completely recover their original structure and function in the eye. For manufacturability, the rod diameter and length must be produced to predetermined specifications for packaging purposes and for providing the customer with consistent product features and handling characteristics. It is clear that a flexible and repeatable mechanism for producing rods of different diameters and length would be preferred for use with thermodynamic intraocular lenses, such as the full-size SMART™ IOL.

In one of the preferred embodiments of the present invention, the SMART™ IOL is deformed into a rod with the crimper device at a temperature equal to or higher than the melting temperature of the lens material. See FIG. 4. The deformed SMART™ IOL, while still inside the crimper device, is placed in an environment chilled to a temperature below the melting temperature of the SMART™ IOL material to allow re-crystallization to occur. Thus, the SMART™ IOL successfully transforms its lens shape into a small diameter solid rod. The rod thus formed may be stored for later use by maintaining the rod below the material's melting temperature or by placing the rod in a form-fitting package that prevents the rod from recovery back to the lens shape if the environment temperature rises to or above the melting temperature of the lens material.

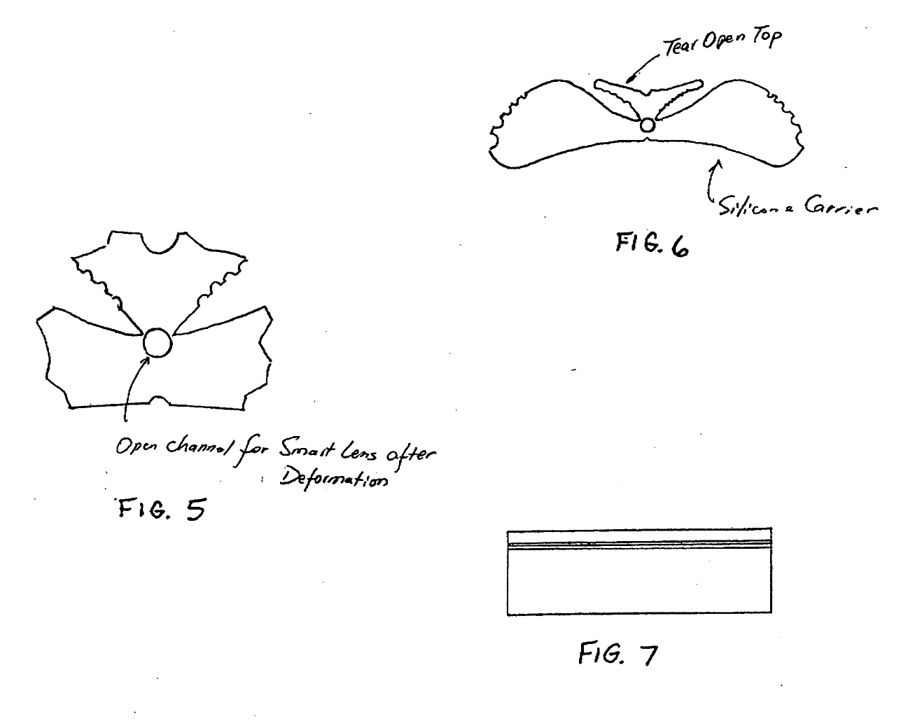

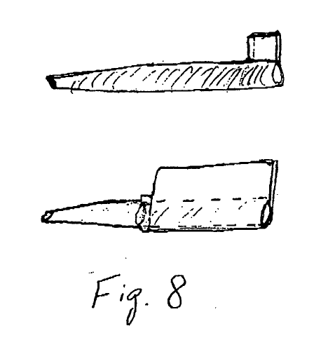

In one of the preferred embodiments of the present invention, the form-fitting package, or a carrier, may include a cylindrical chamber with a diameter closely matching the SMART™ IOL rod diameter. In one example, the carrier is an extruded silicone device with a cylindrical channel. It may have a tear away section that can be removed to retrieve the SMART™ IOL rod. See FIG. 5, FIG. 6 and FIG. 7. The dimension of the open channel is closely matched to the dimensions of the SMART™ IOL rod in order to maintain the rod shape in uncontrolled environments. In an alternate embodiment of the carrier, the rod may be inserted into a thermoplastic cartridge that includes a channel with a diameter that closely matches the rod diameter. The SMART™ IOL may now be exposed to normal temperatures during transportation and shelf storage. The SMART™ IOL rod may be retrieved from the packaging carrier by peeling open the carrier at a temperature below the melting temperature of the lens material or by pushing the rod into a cooled delivery apparatus with a plunger. See FIG. 8.

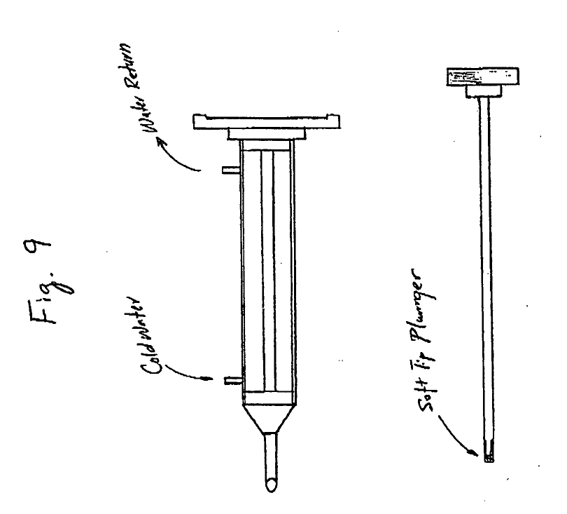

In one of the preferred embodiments, a delivery apparatus with a temperature control is illustrated in FIG. 9. In this inserter design, the central tube has an interior diameter equal to or slightly larger than the diameter of the SMART™ IOL rod. The outside jacket system can be circulated with a cold fluid at a temperature sufficiently lower than the melting temperature of the lens material so that the rod positioned inside the central tube will not prematurely recover back to its lens shape. The cold fluid circulating system may be integrated into a phacoemulsification apparatus, an instrument used to aid in the surgical removal of a human crystalline lens. Alternatively, the jacket can be filled with cold gel or be thermoelectrically cooled to prevent the rod from prematurely changing its shape. In either case, the chilled jacket needs to be cold enough to ensure the rod inside the tube or in the attached cartridge maintains its rod shape. Any premature shape recovery will jam the injector system and may also cause damage to the SMART™ IOL. Additional features of the delivery device of the present invention may also include a sheath surrounding the jacket to isolate the chilled jacket from being warmed up by the surgeon's hands. This sheath also helps the surgeon to hold the device without feeling cold. A plunger mechanism is placed in the back of the inserter for pushing the rod through the injector.

Once the eye is prepared for the lens implantation, a surgeon may slowly push the plunger to deploy the SMART™ IOL into the capsular bag. While the cold SMART™ IOL is deployed, the inserter tip will provide guidance for placing the SMART™ IOL in a desired position inside an eye. The surgeon may also use a soft tip or rounded tip probe through another small incision, a paracentesis, to guide the SMART™ IOL inside the eye without damaging the lens. Once the rod resides in the bag, the human body temperature of the eye warms it up and the rod starts to recover back to its initial biconvex lens shape. The lens delivery process continues until the fully recovered SMART™ IOL fills the bag. The use of the insertion apparatus allows successful injection of SMART™ IOLs through incisions with a smaller diameter than the undeformed lens. The insertion apparatus can be produced from stainless steel, titanium, plastic, glass or a combination thereof.

Methods for processing the SMART™ IOL by deforming its shape, maintaining it in the deformed shape, and reforming back to the original optical shape after inserting the rod through a small incision into the eye are provided and are considered within the scope of the present invention. These methods have, in general, been discussed above, and comprise temperature control and providing an effective amount of time, tools and steps for the thermodynamic intraocular lens shape transformation.

### EXAMPLES

In order that the present invention may be more fully understood, the following examples and other comparative results are given by way of illustration only and are not intended to be limiting.

### Example 1

A full-size intraocular lens of the type described in U.S. Pat. No. 6,679,605 (Zhou, et al), incorporated herein by reference, is produced with an equatorial diameter of 10 mm and a central lens thickness (the distance from the apex of the anterior lens surface to the apex of the posterior lens surface) of 4 mm. The lens is placed on a 0.005 inch thick sheet of polytetrafluoroethylene and coated with a layer of a 10% solution of hydroxypropylmethyl cellulose in water. The sheet is rolled into a tube with the lens at the center of the tube. The tube is placed into a crimper mechanism of the design described in U.S. Pat. No. 6,629,350 (Motsenbocker). The radially distributed arms of the crimper gradually and evenly compress the lens shape into a long, thin rod. The thermoplastic sheet protects the sensitive lens surface from being damaged by the crimper mechanism. The mechanism is submerged into a 0° C. water bath and held there for approximately 5 minutes, allowing the lens polymer to drop below its melting temperature to recrystallize and to solidify. The solid rod, measuring 3 mm in diameter and 37 mm in length, is removed from the crimper and rolled sheet. The rod is stored at temperatures between −20 and +10° C. An injector is produced, consisting of a center tube with an inner diameter of 3 mm surrounded by a cooling jacket with an outer diameter of 15 mm. The plunger has a soft polytetrafluoroethylene tip to seal the barrel of the injector and prevent damage to the lens. The cooling jacket is filled with a gel capable of holding a temperature of 0° C. or below for an extended period. The cold gel temperature is about −10° C. A small quantity of hydroxypropylmethyl cellulose viscoelastic gel is introduced into the injector barrel. The rod is loaded into the barrel and a small quantity of the viscoelastic gel is placed behind the rod. The beveled injector tip is introduced through a 4 mm scleral tunnel incision produced on the corneal surface of an enucleated human cadaver eye. The tip is pushed into the 4 mm diameter capsulorhexsis. The rod is slowly delivered into an aphakic human lens capsule, where it recovers to its original dimensions and full-size lens shape.

### Example 2

A full-size intraocular lens of the type described in U.S. Pat. No. 6,679,605 (Zhou, et al) is produced with an equatorial diameter of 10 mm and a central lens thickness (the distance from the apex of the anterior lens surface to the apex of the posterior lens surface) of 2.2 mm. The lens is placed on a 0.005 inch thick sheet of polytetrafluoroethylene and coated with a layer of a 10% solution of hydroxypropylmethyl cellulose in water. The sheet is rolled into a tube with the lens at the center of the tube. The tube is placed into a crimper mechanism of the design described in U.S. Pat. No. 6,629,350 (Motsenbocker). The radially distributed arms of the crimper gradually and evenly compress the lens shape into a long, thin rod. The thermoplastic sheet protects the sensitive lens surface from being damaged by the crimper mechanism. The deformed lens, while still inside the crimper, is submerged into a 0° C. water bath and held there for approximately 5 minutes, allowing the lens polymer to drop below its melting transition temperature and solidify. The solid rod, measuring 2 mm in diameter and 35 mm in length, is removed from the crimper and rolled sheet. The rod is stored at temperatures between −20 and +10° C. An injector is produced, consisting of a center tube with an inner diameter of 2.02 mm surrounded by a cooling jacket with an outer diameter of 15 mm. The plunger has a soft silicone tip to seal the barrel of the injector and prevent damage to the lens. The cooling jacket is filled with a gel (about −10° C.) capable of holding a temperature of 0° C. for an extended period. A small quantity of hydroxypropylmethyl cellulose viscoelastic gel is introduced into the injector barrel. The rod is loaded into the barrel and a small quantity of the viscoelastic gel is placed behind the rod. A soft polypropylene cylindrical tip with an inner diameter of 2.2 mm and an outer diameter of 2.4 mm is slipped over the first 15 mm of the injector barrel. The polypropylene tip extends approximately 12 mm beyond the injector barrel. The small tip size allows introduction of the lens through a small incision. The beveled injector tip is introduced through a 3 mm scleral tunnel incision produced on the corneal surface of an enucleated human cadaver eye. The cataractous human crystalline lens has been removed by phacoemulsification. The tip is pushed into the 3.0 mm diameter capsulorhexsis. The rod is slowly delivered into the aphakic human lens capsule, where it warms up, recovering to its original dimensions and full-size lens shape as it is being introduced into the capsule.

### Example 3

A three-piece IOL is prepared from the same composition as Example 1 of the U.S. Pat. No. 6,679,605 (Zhou, et al). The lens is warmed at 50° C. for about one minute and then placed on a 0.005 inch thick sheet of polytetrafluoroethylene and coated with a layer of a 10% solution of hydroxypropylmethyl cellulose in water. The sheet is rolled into a tube with the lens at the center of the tube and with the two haptics at each end of the roll. The tube is placed into the same crimper as described above, and after following similar steps as in Example 1, a rod with a diameter of about 1.5 mm and length of about 11 mm is obtained. This whole rod is successfully delivered into a capsular bag through a 2 mm incision in a cadaver human eye. Once warm saline is introduced, it recovers back to its original three-piece lens design inside the capsule.

1. A method for insertion of a thermodynamic shape memory intraocular lens in an eye, comprising the steps of:
(a) placing said lens into a deformation device;
(b) forming said lens into a rod having a predetermined cross-sectional diameter of from about 1 to about 4 mm using said deformation device;
(c) inserting said lens, configured as a rod, into the eye through an incision.

2. The method according to claim 1 wherein the lens is made from a polymer composition having a glass transition temperature of 20° C. or lower, and a melting temperature of from about 1° C. to about 37° C.

3. The method according to claim 2 wherein, following formation of the lens into a rod in step (b), the temperature of the lens is lowered to below its melting or glass transition temperature.

4. The method according to claim 3 wherein the temperature of the rod is maintained at below its melting point until after the rod has been inserted into the eye in step (c).

5. The method according to claim 1 wherein a thermoplastic sheet is placed over the optic surface of the lens prior to step (a).

6. The method according to claim 5 wherein the thermoplastic sheet is made from a material selected from polytetrafluoroethylene, polyethylene, polypropylene, polysiloxane, and mixtures thereof.

7. The method according to claim 1 wherein a viscoelastic lubricant is applied to the optic surface of the lens prior to step (a).

8. The method according to claim 7 wherein the viscoelastic lubricant is selected from an aqueous solution of sodium hyaluronate, an aqueous solution of hydroxypropylmethyl cellulose, an aqueous solution of chondroitin sulfate, and mixtures thereof.

9. The method according to claim 3 wherein, after step (b), the rod is placed in a form-fitting package which prevents the rod from recovery back to lens shape.

10. The method according to claim 1 wherein the deformation device is a segmented radial compression device.

11. The method according to claim 1 wherein step (c) is carried out using an inserter which comprises a central channel to hold the lens in rod form; a sleeve surrounding the central channel, said sleeve cooled to keep the lens at a temperature below its melting temperature; and a plunger used to push the lens out of the central channel and into the eye.

12. The method according to claim 11 wherein the inserter additionally comprises an insulating sheath surrounding the cooled sleeve.

13. A segmental radial compression device holding a thermodynamic shape memory intraocular lens.

14. The device according to claim 13 wherein the lens is made from a polymer composition having a glass transition temperature of 20° C. or lower, and a melting temperature of from about 1° C. to about 37° C.

15. A device for inserting an intraocular lens in the form of a rod through an incision into the eye, comprising a central channel to hold the lens in rod form; a sleeve surrounding the central channel, said sleeve cooled to keep the lens at a temperature below its melting temperature; and a plunger used to push the lens out of the central channel and into the eye.

16. The device according to claim 15 which additionally comprises an insulating sheath surrounding the coolant-containing sleeve.

17. The device according to claim 15 made from materials selected from stainless steel, titanium, plastic, glass, and combinations thereof.
